SEQUENCE LISTING (3 sequences)

SEQ ID NO: 1 - protein, 11 aa, Measles virus
  MISC_FEATURE: amino acids 411-421
GPRQAQVSFLQ

SEQ ID NO: 2 - protein, 18 aa, Measles virus
  MISC_FEATURE: amino acids 489-506
RRSAEPLLRLQAMAGISE

SEQ ID NO: 3 - protein, 10 aa, Measles virus
  MISC_FEATURE: amino acids 516-525
TVYNDRNLLD

(19) United States
(12) Patent Application Publication
(10) Pub. No.: US 2004/0091501 A1
(43) Pub. Date: May 13, 2004

(54) Immunosuppresor agent derived from morbillivirus

(76) Inventors: Branka Horvat, Saint Genis Laval (FR); Julien Marie, Brives-Charensac (FR); Jeanne Kehren, Bantzenheim (FR); Helene Valentin, Bron (FR); Chantal Rabourdin-Combe, Sainte-Foy-Les-Lyon (FR); Marie-Claude Biemont, Lyon (FR)

Correspondence Address: MORGAN LEWIS & BOCKIUS LLP, 1111 PENNSYLVANIA AVENUE NW, WASHINGTON, DC, 20004, US

(21) Appl. No.: 10/311,269

(22) PCT Filed: Jun. 15, 2001
(86) PCT No.: PCT/EP01/06772

(30) Foreign Application Priority Data
Jun. 14, 2000 (FR) 0007587

(51) Int. Cl. A61K 39/12

(52) U.S. Cl. 424/186.1

(57) ABSTRACT

The invention relates to the use of morbillivirus nucleoproteins, or fragments thereof comprising the C-terminal portion of 100 to 130 amino acids for the preparation of immunosuppressor drugs, and to the use of morbilliviruses within which the C-terminal portion has been deleted or altered, for the preparation of vaccines.

The present invention relates to immunosuppressive agents derived from morbilliviruses.

The genus morbillivirus, of the family Paramyxoviridae, in particular comprises the measles virus (MV), the canine distemper virus (CDV), the rinderpest virus and the pest des petites ruminants virus (PPRV). They exhibit a strict infectious tropism. For example, the measles virus infects only humans and a few large monkey species.

Morbilliviruses are enveloped viruses. The viral envelope, which is lipoprotein in nature, comprises two glycoproteins, hemagglutinin (HA) and the fusion (F) protein composed of two subunits F₁ and F₂. The HA protein is involved in binding of the virus to the host-cell receptor. In humans, the MV receptor has been identified, at least in the case of the vaccinal strains, as being the CD46 membrane glycoprotein, which is expressed on all cells except erythrocytes.

The viral genome, consisting of a negative-state single-stranded RNA, is protected by a nucleocapsid, a helical ribonucleoprotein particle. The nucleocapsid is composed of three proteins: the nucleoprotein (NP) directly associated with the viral RNA and the other two proteins, also associated with the RNA, the phosphoprotein (P) and the L protein, involved in replication and transcription of the viral genome. The cohesion of the envelope-nucleocapside assembly is provided by the matrix protein (M) located at the interface of the nucleocapside and the viral envelope.

The existence of immunosuppressive effects resulting from infection with MV has been known for a long time. It was described for the first time in 1908 by VON PIRQUET, who reported that the majority of patients suffering from measles exhibited a transient decrease in the intensity of the delayed hypersensitivity skin reaction in tuberculin tests [VON PIRQUET, Dtsch. Med. Wochenschr., 30, 1297-1300, (1908)]. These immuno-suppressive effects have since been confirmed by other investigators [TAMASHIRO et al.; Pediatr. Infect. Dis., 6, 451-454 (1987); BECKFORD et al., S. Afr. Med. J., 68, no. 12, 858-863 (1985); HIRSCH et al., Clin. Immunol. Immunopath., 31, 1-12, (1984)], and it is acknowledged that, by promoting the development of secondary infections, they constitute a major cause of death subsequent to infections with MV.

In addition, this immunosuppression has also been observed, although to a lesser degree, in the case of measles vaccination, where live attenuated strains of the measles virus are used [FIREMAN et al., Pediatrics, 43, 264-272, (1969); WEISS, Science, 258, 546-547, (1992); HUSSEY at al., J. Infect. Dis., 173, 1320-1326, (1996); BOUCHAUD et Mouas, Ann. Med. Interne, 149, no. 6, 351-360, (1998)].

It has been observed that T lymphocytes derived from the circulating blood of patients infected with a pathogenic strain or the vaccinal strain of MV exhibit a decrease in the ability to proliferate in response to mitogens, allergens or booster antigens [COOVADIA et al., Arch. Dis. Child, 53, no. 11, 861-867, (1978); HIRSCH et al., cf. above; BORROW and OLDSTONE, Curr. Top. Microbiol. Immunol., 191, 85-100, (1995); HUSSEY et al., cf. above].

The mechanisms which direct the immunosuppressive effects of Morbilliviruses, and in particular of MV, are still poorly understood. In fact, because of the lack of an experimental animal model easy to study, most observations regarding these mechanisms have been made in vitro.

It has thus been observed that MV induces death by apoptosis of infected immune cells such as macrophages [ESOLEN et al., J. Virol., 69, no. 6, 3955-3958, (1995)], thymic epithelial cells [VALENTIN et al., J. Virol. 73, no. 3, 2212-2221, (1999)] and dendritic cells derived from monocytes [FUGIER-VIVIER et al., J. Exp. Med., 186, no. 6, 813-812, (1997); SCHNORR et al., Proc. Natl. Acad. Sci. USA, 94, no. 10, 5326-53331, (1997)] or from umbilical cord blood [GROSJEAN et al., J. Exp. Med., 186, no. 6, 801-812, (1997)], and also a loss of the lymphoproliferative response to mitogens and arrest of the cell cycle of T lymphocytes in the Go/G₁ phase [McCHESNEY et al., J. Immunol., 140, no. 4, 1269-1273, (1988); NANICHE et al., J. Virol., 73, no. 3, 1894-1901, (1999)]. Inhibition of antibody production by B lymphocytes infected in vitro has also been observed [GALAMA et al., Cell Immuno., 50, no. 2, 405-415, (1980); CASALI et al., J. Exp. Med., 159, no. 5, 1322-1337, (1984)], as has a disturbance of cytokine production by T lymphocytes and antigen-presenting cells [GRIFFIN, Curr. Top Microbiol. Immunol., 191, 117-134, (1995); LEOPARDI et al., J. Immunol., 149, no. 7, 2397-2401, (1992).

The viral components involved in these various effects still remain poorly understood.

SCHLENDER et al. [Proc. Natl. Acad. Sci. USA, 93, no. 23, 13194-13199, (1996)] and NIEWIESK et al. [J. Virol., 71, 7214-7219, (1997)] report that MV or UV-inactivated MV (MV-UV) induces, in vitro, a loss of mitogen-induced T lymphocyte proliferation; this effect is thought to involve the envelope proteins H and F.

It has also been observed that the interaction of UV-inactivated MV with the CD46 molecule inhibits IL-12 secretion by monocytes [KARP et al., Science, 273, no. 5272, 228231, (1996)].

Finally, RAVANEL et al., [J. Exp. Med., 186, 269-278, (1997)] report that the NP protein affects B lymphocytes functions by inhibiting the immunoglobulin production thereof [RAVANEL et al., J. Exp. Med., 186, no. 2, 269-278, (1997)].

It therefore appears that current available knowledge of the immunosuppressive effects of morbilliviruses is still very fragmented. In addition, the available results resulting mainly from in vitro studies on isolated cell populations only give an incomplete representation of the complexity of the physiological interactions resulting in the immuno response.

A more complete knowledge of the mechanism(s) involved in morbillivirus-induced immunosuppression may in particular make it possible to obtain novel immunosuppressive treatments, or else to prepare morbillivirus vaccines lacking immunosuppressive effects or exhibiting only attenuated immunosuppressive effects.

The inventors have now developed a murine experimental model for studying morbillivirus-induced immuno-supppression.

They have thus been able to observe that, even in the absence of any viral replication, morbillivirus proteins induce inhibition of the cell-mediated immune response (type IV reactions according to the GELL and COOMBS classification), according to 2 different modes:

1) inhibition of CD8-dependent contact hyper-sensitivity;

2) inhibition of CD4-dependent, tuberculin-type delayed hypersensitivity.

They have also noted that, surprisingly, this inhibition of the cell mediated immuno response mainly involves the NP protein, and more particularly a region corresponding to approximately 125 C-terminal amino acids of this protein, and that it does not require the involvement of the CD46 cellular receptor. The envelope glycoproteins F and H, and also the CD46 receptor, increase, however, the immunosuppressive effects.

The inventors have also observed that morbilliviruses can induce immunosuppression in a host which is different from their natural host, which shows that the mechanisms directing the immunosuppression induced by these viruses are not species specific.

A subject of the present invention is the use of a preparation of proteins comprising at least one morbillivirus nucleoprotein, or a fragment thereof comprising all or part of the approximately 100 to 130 amino acid C-terminal region of said nucleoprotein, for producing a medicinal product which inhibits the cell-mediated immune response.

According to one embodiment of the present invention, said fragment comprises at least one sequence of said C-terminal region which is conserved between the nucleoproteins of various morbilliviruses, and in particular between the nucleoproteins of the morbilliviruses MV, CDV and PPRV.

Although said C-terminal regions represents the portion of the nucleoprotein for which the sequence diverges most from one morbillivirus to the other, there are, nevertheless, within this region, several very conserved portions (cf. for example DIALLO et al. [J. Gen. Virol., 75, 233-237, (1994)].

Among the fragments of said C-terminal region corresponding to conserved portions, which can be used in accordance with the invention, mention will be made in particular of:

a fragment comprising amino acids 411-421 of the peptide sequence of the measles virus nucleoprotein, or the corresponding sequence of the nucleoprotein of another morbillivirus;

a fragment comprising amino acids 489-506 of the peptide sequence of the measles virus nucleoprotein, or the corresponding sequence of the nucleoprotein of another morbillivirus;

a fragment comprising amino acids 516-625 of the peptide sequence of the measles virus nucleoprotein, or the corresponding sequence of the nucleoprotein of another morbillivirus.

The positions indicated above refer to the numbering of the amino acids in the sequences published by DIALLO et al. [cf. above].

Advantageously, a preparation of proteins which can be used in accordance with the invention also comprises the envelope glycoproteins F and H of a morbillivirus.

A preparation which can be used in accordance with the invention may, for example, be obtained by inactivation of a morbillivirus, in particular by treatment with ultraviolet rays; it may also be obtained using the proteins, or fragments thereof, mentioned above.

The morbillivirus proteins, and also the fragments thereof, which can be used in accordance with the invention are known in themselves; they may be prepared by conventional methods, for example by purification from viral cultures, or advantageously by genetic engineering or by peptide synthesis.

The inhibition of cell-mediated immunity induced by the preparations of morbillivirus proteins is comparable to that obtained in a treatment with a high dose of anti-inflammatories, or based on powerful immunosuppressants, such as dexamethosone.

The present invention may be implemented in the context of the prevention or treatment of all pathological conditions in which it is desirable to inhibit the cell-mediated immuno response. Mention will, for example, be made of inflammatory pathological conditions resulting in particular from infection with a pathogenic agent (leprosy, tuberculosis, leishmaniasis, listeriosis, candidiasis, etc.) or allergic contact dermatitis induced by various sensitizing agents. It may also be implemented in the prevention or treatment of transplant rejection, or of graft versus host disease.

The present invention may also be used in the context of the development of novel vaccines, in particular of measles vaccines, lacking immunosuppressive effects or having attenuated immunosuppressive effects. These vaccines may in particular be obtained from morbilliviruses in which all part of the nucleoprotein, and in particular the approximately 110 to 130 amino acid C-terminal region of said nucleoprotein, has been deleted or altered. It may be altered in particular by one or more modifications in at least one of the sequences conserved between the various morbilliviruses, and/or by deletion of one or more of said conserved sequences. Advantageously, said modification or said deletion concerns at least one of the sequences 411-421, 489-506 or 516-625, mentioned above.

A subject of the present invention is also methods for evaluating the immunosuppressive potential of a preparation of a morbillivirus, for example of a morbillivirus vaccine, or of a preparation which can be used as an immunosuppressive in accordance with the invention.

According to a first variant of the present invention, said immunosuppressive potential is evaluated by determining the amount of NP protein of said morbillivirus present in said preparation.

According to a preferred embodiment of this first variant, said method comprises bringing said preparation into contact with at least one antibody against said NP protein, and preferably at least one antibody against an epitope carried by the approximately 110 amino acid C-terminal region of said NP protein, and quantifying the antibody/antigen complex formed, by any suitable means known themselves to those skilled in the art.

According to a first variant of the present invention, said immunosuppressive potential is evaluated using mice sensitized to DNFB or to KLH.

Advantageously, and in particular to evaluate the immunosuppressive potential of a preparation comprising the morbillivirus envelope proteins H and F, said mice are transgenic mice expressing the CD46 receptor. Transgenic mice expressing the CD46 receptor are known in themselves; they may, for example, be obtained as described by THORLEY et al. [Eur. J. Immunol., 27, 726-734, (1997)] or by EVLASHEV et al. [J. Virol., 74, 1373-1382, (2000)].

The present invention will be more clearly understood from the further description which follows, which refers to nonlimiting examples illustrating the inhibitory effects, in vivo in a murine model, of various morbillivirus preparations on delayed hypersensitivity reactions.

### EXAMPLE 1

### Inhibition of Delayed Hypersensitivity Reactions in Mice by UV-Inactivated Measles Virus

The effect of UV-inactivated measles virus on the cell-mediated immune response was tested in vivo, in mice, using models representative of the 2 aspects of delayed hypersensitivity: contact hypersensitivity to DNFB, and delayed hypersensitivity to KLH, representative of tuberculin-type hypersensitivity.

7- to 11-week-old C57BL/6 mice (IFFA CREDO) were used for these experiments.

The Edmonston MV (ATCC VR-24) and, as control, another paramyxovirus, the respiratory syncitial virus (RSV, Group A strain Long), were used. The viruses were propagated separately on monkey kidney fibroblast (Vero) cells. After mechanical lysis of the cells, carried out when 50% of them exhibited a cytopathic appearance, the cell lysate containing the infectious viral particles was frozen at −80° C. and thawed, then centrifuged and, finally, stored at −80° C. Each batch of virus was inactivated by UV exposure (254 nm) for 45 min at 4° C. The loss of infectious nature was tested on Vero cells.

A “dummy” preparation consisting of the noninfected Vero cell culture supernatant was used as a control.

Intraperitoneal injections of MV were given either to wild-type mice or to transgenic mice expressing the CD46 molecule.

The various preparations were injected (volume of an injection: 500 μl) into the peritoneal cavity (IP) of mice, in a proportion of 5×10⁶ viral particles for the RSV inactivated by UV exposure (RSV-UV), and of 106 or 5×10⁶ viral particles for the MV inactivated by UV exposure (MV-UV).

DNFB Contact Hypersensitivity Test

DNFB (2,4-dinitrofluorobenzene, SIGMA) was diluted in acetone, olive oil (4:1) immediately before use. The DNFB contact hypersensitivity (CHS) reaction was produced as described according to GARRIGUE et al., [Contact Dermatitis, 30, no. 4, 231-237, (1994)]. Six hours after injection of the viral preparation or of the dummy preparation, 24 μl of a 0.5% DNFB solution were applied over 2 cm² of preshaved ventral skin. After 5 days, the sensitized animals, and the nonsensitized animals from a control group, received 10 μl of nonirritant solution of DNFB distributed over each of the faces of the right ear and 10 μl of acetone-olive oil solution over the left ear. The thickness of the ears was measured with a micrometer (J15; Bell SA, France) before application and each day after application. The edema of the ear is calculated using the following formula: [(T−T₀)right ear]−[(T−T₀)left ear], where T and To are respectively the values of the thickness of the ear before and after elicitation.

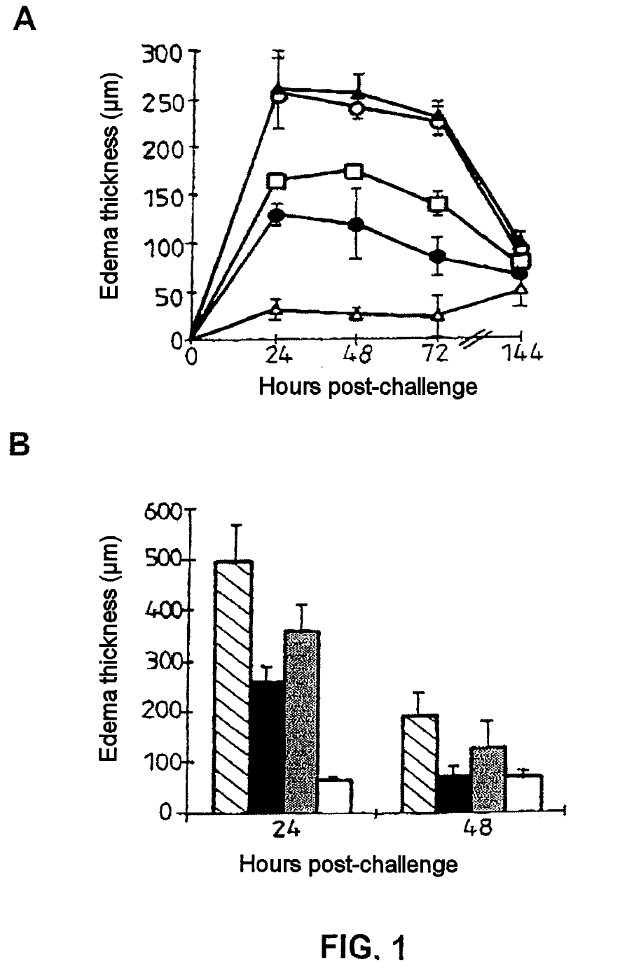

The results are given in FIG. 1(a), which represents the mean size of the edema of the ear at various times following application.

Legend of FIG. 1(a):

▴: RSV-UV

●: 5×10⁶ MV-UV

□: 10⁶ MV-UV

◯: dummy preparation

Δ: nonsensitized mice

KLH Delayed Hypersensitivity Test

The delayed hypersensitivity was measured using a conventional test for measuring foot pad edema. Six hours after injection of the viral preparation or the dummy preparation, the mice were sensitized by subcutaneous injection of 300 μg of KLH (keyhole limpet hemocyanin, SIGMA) emulsified in complete Freund's adjuvant. 7 days later, a second injection (150 μg of KLH in a PBS buffer) was given in the pads of the left foot of the sensitized animals, and of the nonsensitized animals from a control group; PBS alone was injected into the right foot. Pad thickness was measured 24 hours before and 48 hours after the boost injection. The pad edema is calculated using the following formula: [(T−T₀)right pad]−[(T−T₀)left pad], where T and T₀ are respectively the values of the pad thickness before and after the boost injection. The results are given in FIG. 1(b), which represents the mean size of the pad edema 24 hours after the boost injection.

Legend of FIG. 1(b):

Hatched bars: dummy preparation

Black bars: 5×10⁶ MV-UV

Grey bars: 10⁶ MV-UV

White bars: nonsensitized mice

These results show that the mice injected with MV-UV develop a delayed hypersensitivity response which, regardless of whether it is contact hypersensitivity or tuberculin-type hypersensitivity, is approximately two-fold less than the control mice injected with the dummy preparation.

Under the same conditions, control mice injected with RSV-UV develop a delayed hypersensitivity response which is equivalent to that of the control mice injected with the dummy preparation.

The MV therefore induces immunosuppression in vivo, this being in the absence of any viral replication.

### EXAMPLE 2

### Role of the Measles Virus Nucleoprotein (NP) in the Inhibition of Delayed Hypersensitivity Reactions in Mice

To study the contribution of the NP in immunosupppressive effect induced by MV-UV in mice, C57BL/6 mice or mice lacking the FcγR receptor (FcγRUU^{-/-}×FcγR^{-/-}; 0005856MM; TACONIC, USA) were injected intraperitoneally with purified recombinant NP.

The recombinant nucleoprotein was prepared according to the following protocol:

Spodoptera frugiperda Sf-9 cells were infected, in a proportion of 1 PFU per cell, with a recombinant baculovirus (AcNPVNP) comprising the coding sequence of the MV NP. After culturing for three days, the cells were collected by centrifugation (250 g, 5 min, 4° C.) and the cell product was washed in PBS buffer. The cells were then placed in a hypotonic buffer (10 mM tris-HCl, pH 7.5; 10 mM NaCl and lysed by adding a nonionic detergent, 1% Nonidet P-40. In order to avoid problems of degradation, protease inhibitors were added at the time of cell lysis and the lysates were kept at 4° C. In order to remove the nuclei, the lysates were centrifuged at 1000 g, and then ultracentrifugation (1000 g, 10 min) in the presence of 10 mM EDTA was carried out, thus removing the cell debris. The supernatant recovered was centrifuged in CsCl for 2 hours at 36000 rpm at 12° C., with an SW41 rotor (BECKMAN). This gradient is made up of steps of 2 ml of 40% CsCl (W/V), 2 ml of 30% CsCl, 2 ml of 25% CsCl and 2 ml of 5% sucrose (W/V). The NP which forms a visible band at the density of 30% of the CsCl was recovered by drawing it up, diluted in PBS and centrifuged for 2 hours at 36000 rpm. The final pellet was resuspended in PBS. Each batch was tested, by protein assay, (BCA kit PIERCE), by acrylamide gel and by Western Blotting.

For each experiment, 100 μg of NP, 5×10⁶ MV-UV particles, or 500 μl of the dummy preparation were injected into the peritoneal cavity of the mice, according to the protocol described in example 1 above.

These animals were then sensitized to DNFB or to KLH, as described in example 1 above.

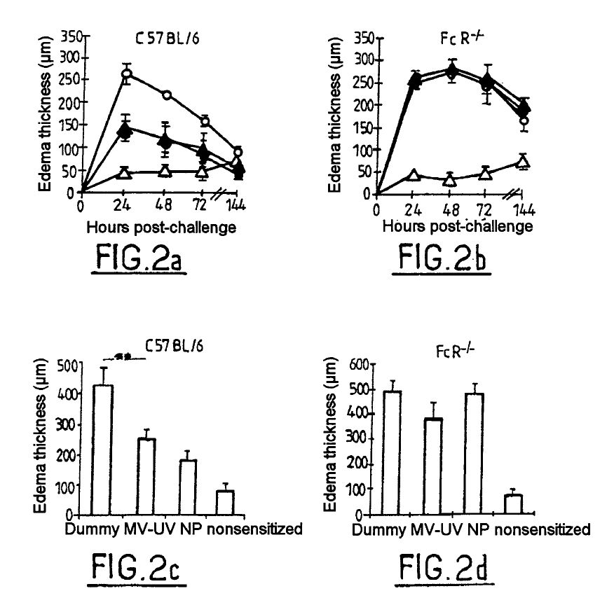

The results are given in FIGS. 2(a) to 2(d).

FIG. 2(a): DNFB hypersensitivity test in C57BL/6 mice

●: 5×10⁶ MV-UV

♦: recombinant NP

◯: dummy preparation

Δ: non-sensitized mice

FIG. 2(b): DNFB Hypersensitivity Test in FcγR^{-/-} Mice

●: 5×10⁶ MV-UV

♦: recombinant NP

◯: dummy preparation

Δ: non-sensitized mice

FIG. 2(c): KLH Hypersensitivity Test in C57BL/6 Mice

FIG. 2(d): KLH Hypersensitivity Test in FcγR^{-/-} Mice

While the mice injected with the dummy preparation exhibit an edema characteristic of the DNFB hypersensitivity reaction, the NP induces, in the C57BL/6 mice, an inhibition of the hypersensitivity reaction to DNFB similar to that engendered by the MV-UV. Additional experiments carried out with higher concentrations of NP show that this effect is dose-dependent; the inhibition induced by injecting 40 μpg of NP is slightly less than that observed after injecting 100 μg, and no inhibition is observed after injecting 4 μg of NP.

In the case of the KLH hypersensitivity reaction, the inhibition induced by the NP is comparable to that induced by the MV-UV, although a little stronger.

On the other hand, in the mice lacking the FcγR, no immunosuppressive effect of the NP or of the MV-UV is observed in the case of the DNFB hypersensitivity reaction. In the case of the KLH hypersensitivity reaction, no immunosuppressive effect on the NP is observed, and a weak immunosuppressive effect on the MV-UV is observed.

### EXAMPLE 3

### Role of the Measles Virus Envelope Glycoproteins HA and F in the Inhibition of Delayed Hypersensitvity Reactions in Mice

To analyze the role of the MV envelope proteins in the immunosuppression induced in mice, a recombinant measles virus lacking the envelope glycoproteins HA and F was used, either on C57BL/6 mice or on transgenic mice expressing the CD46 molecule.

The mice transgenic for CD46 were produced by injecting the CDNA of the human gene encoding the molecular isoform CD46 BC Cyt-1 under the control of the CD46 promoter, into mouse ovocytes. These mice express the CD46 molecule ubiquitously, in the H-2b genetic background [THORLEY et al., Eur. J. Immunol., 27, 726-734, (1997)].

The Edmonston tag (EDtag) MV is a recombinant MV derived from the Edmonston strain by molecular cloning of the entire genome [TOBER et al., J. Virol., 72, no. 10, 8124-8132, (1998)]. The MGV MV is a recombinant MV lacking the envelope glycoproteins HA and F [SPIELHOFER et al., J. Virol. 72, no. 3, 2150-2159, (1998)].

The MV strains were propagated, harvested, and UV-inactivated according to the protocol described in example 1 above.

For each experiment, 5×10⁶ MV-UV, EDtag-UV or MGV-UV particles, or 500 μl of dummy preparation, were injected into the peritoneal cavity of the mice, according to the protocol described in example 1.

The animals were then sensitized to DNFB as described in example 1.

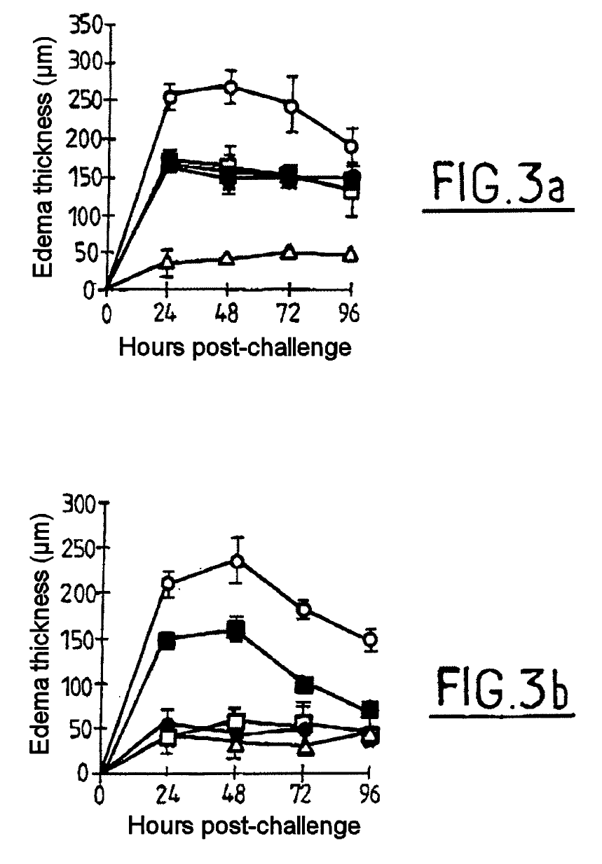

The results are given in FIG. 3(a), for the C57BL/6 mice, and 3(b) for the mice transgenic for CD46.

Legend of FIGS. 3(a) and 3(b):

●: MV-UV

□: EDtag-UV

▪: MGV-UV

◯: Dummy preparation

Δ: Non-sensitized mice

In the C57BL/6 mice, or in the mice transgenic for CD46 which had received an injection of MGV-UV, an inhibitory effect of equivalent intensity is observed. In both cases, this effect is comparable to that resulting from the injection of 100 μg of purified NP (results not shown).

In the C57BL/6 mice, the inhibitory effect of MGV-UV is also comparable to that engendered by the MV-UV or the control recombinant virus EDtag-UV.

On the other hand, in the mice transgenic for CD46, the inhibitory effect of MV-UV and of EDtag-UV is greater than that of MGV-UV in the inhibition of the CHS reaction DNFB. The injection of MGV-UV induces only partial inhibition of the edema, whereas injection of MV-UV or EDtag-UV completely inhibits the formation of the edema.

These results show that, although the viral envelope glycoproteins H and F are not essential for the induction of inhibition of the hypersensitivity reaction, they make it possible, in the presence of the CD46 receptor, to increase the immunosuppressive effect, until this reaction is completely inhibited.

Further experiments, in which the sensitization with DNFB was carried out two weeks after the injection of viral preparation, were carried out. Under these conditions, an inhibitory effect on the hypersensitivity reaction was observed, for the viral preparations containing the glycoproteins H and F, in the case of the mice transgenic for CD46, which indicates that expression of this receptor may make it possible to increase the duration of the immunosuppressive effect of the viral proteins.

### EXAMPLE 4

### Effect of UV Inactive Measles Virus on the Cytotoxic Actvity of Lymphocytes (CTLS) Specific for Hapten

In order to analyse the cytotoxic activity induced after sensitization, the spleen cells of C57BL/6 mice, and mice transgenic for CD46, injected or not injected with MV-UV or NP, and sensitized to DNFB, were tested for their ability to lyse EL4 target cells coupled to DNBS (2,4-dinitrobenzenesulfonic acid; SIGMA).

For each experiment, 100 μg of NP, 5×10⁶ MV-UV particles, or 500 μl of dummy preparation were injected into the peritoneal cavity of the mice, and the animals were then sensitized to DNFB, according to the protocol described in example 1 above.

The spleen of each animal was removed and a cell suspension was prepared by grinding the organs between sintered glass slides. The splenocytes were washed and the red blood cells were lysed in the presence of 0.83% NH₄Cl for 5 minutes at 37° C.

Splenocytes were isolated in the same way from naïve mouse spleen, and then irradiated at 1500 rad and loaded with hapten by incubation in a 4 mM DNBS solution in order to play the role of APCs.

The splenocytes of the sensitized mice and the hapten-loaded splenocytes were cocultured in RPMI 1640, 10% FCS, in a proportion of 2×10⁶ cells per ml. After culturing for 5 days, the splenocytes restimulated by the culture in the presence of the APCs constitute the effector population for the test.

EL-4 cells (10⁶ cells per ml), acting as target cells, were cultured in RPMI 1640, 10% FCS, and in the presence of tritiated thymidine (10 μCi per ml of culture medium for 3 hours at 37° C.), and then washed twice in RPMI 1640, 10% FCS. A portion of the tritium-labeled EL-4 cells was then loaded with DNBS as described above. The target cells were distributed into 96-well plates in a proportion of 2×10⁴ cells per well. The effector cells were also distributed into the same wells, according to a decrease in target/effector ratio. The control wells (spontaneous lysis) contain only target cells. After coculturing for 4 hours, the cells were transferred onto filters, and the number of counts per minute was counted in the presence of scintillation fluid (WALLAC counter).

The percentage cytotoxicity is determined using the formula:

% cytotoxicity=100(S−E)/S

where S corresponds to the spontaneous lysis (tritiated and haptenized EL-4 without effector cells), and E corresponds to the experimental lysis (tritiated and haptenized EL-4 in the presence of effector cells).

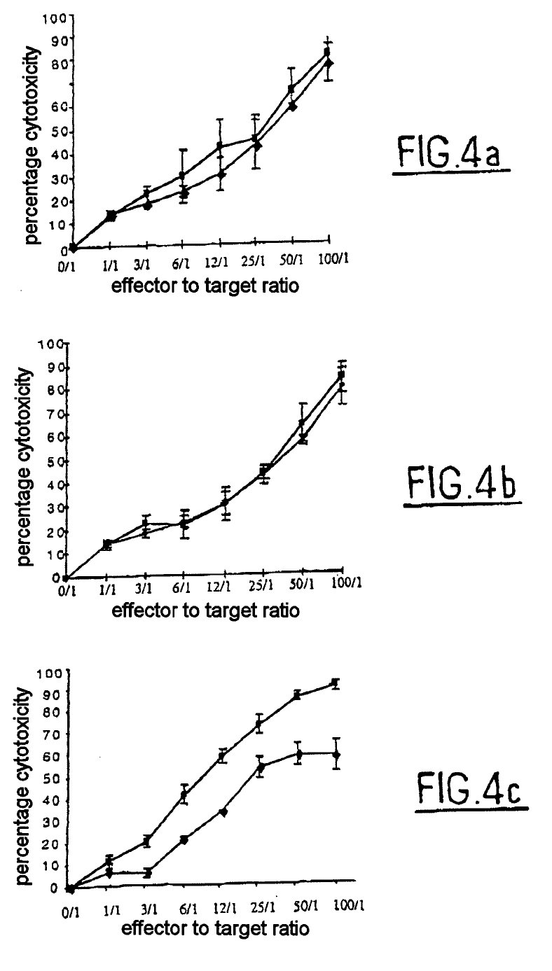

The results are given in FIGS. 4(a) to 4(c).

FIG. 4(a): cytotoxic activity of the cells of C57BL/6 mice injected with MV-UV

♦: MV-UV

▪: dummy preparation

FIG. 4(b): DNFB hypersensitivity test in C57BL/6 mice injected with recombinant NP

●: recombinant NP

▪: dummy preparation

FIG. 4(c): KLH hypersensitivity test in the CD46 mice injected with MV-UV

♦: MV-UV

▪: dummy preparation

As shown in FIGS. 4A and 4B, injecting C57BL/6 mice with MV-UV or with NP does not affect the CTL activity compared with that of the control mice (dummy preparation). On the other hand, injecting MV-UV significantly inhibits the CTL activity of the lymphocytes originating from transgenic CD46 mice (p<0.05) (FIG. 4C). This lack of CTL activity therefore appears to be dependent on the expression of the CD46 molecule.

### EXAMPLE 5

### Effects of UV-Inactivated Measles Virus and of NP on the Proliferation of CD8 T Lymphocytes Specific for the Hapten

Since CD8 T lymphocytes (CD8LTs) constitute the effector population involved in contact hyper-sensitivity to DNFB [KEHREN et al., J. Exp. Med., 189, no. 5, 779-786, (1999)], the question of whether the inhibition of the CSH reaction to DNFB induced by MV-UV and NP was the result of a lack of activation of the CD8LTs during the sensitization phase was investigated. Experiments were carried out on C57BL/6 mice, on transgenic mice expressing the CD46 molecule, or on mice lacking the FcγR receptor.

For each experiment, 100 μg of NP, 5×10⁶ MV-UV particles, 500 μl of dummy preparation or 500 μl of PBS buffer were injected into the peritoneal cavity of the mice, and the animals were then sensitized to DNFB according to the protocol described in example 1 above.

The CD8LTs of each mouse, derived from the lymph nodes draining the area of application of the DNFB, were isolated according to the following protocol:

The lymph nodes draining the area of application of the hapten (inguinal, brachial and axillary lymph nodes) of each animal were removed 4 days after sensitization of the animals, and placed in RPMI 1640, 10% FCS. The lymph nodes were ground between sintered glass slides and the extracted cells were washed and then placed in an incubator at 37° C. for two hours in order to remove the macrophases by adhesion onto plastic. The cells were then incubated for 30 min at 37° C. in the presence of rat moleclonal antibodies: GK1.5 (anti-CD4), M1/70 (anti-macrophage), RA3-6B2 (anti-B220), M5/114 (anti-class II), RB6-8C5 (anti-granulocyte). In order to remove the excess antibody not bound to the cells, the cells were washed in RPMI 1640, 10% FCS. The cells were then incubated for 30 min at 4° C. with shaking, in the presence of magnetic beads coupled to an immunoglobulin against rat antibodies (BIOMAG). The cells bound to the antibodies, recognized by the immunoglobulins present at the surface of the beads, were removed by passing the suspension over a magnet. The purification of the lymphocytes was verified by labeling of the negatively selected cells with an anti-CD8 antibody and analysis by flow cytometry. The cell population thus negatively selected consisted of more than 95% CD8 T lymphocytes.

The CD8LTs thus isolated were cocultured for three days in the presence either of APCs prepared from splenocytes of naïve mice, coupled to DNBS or coupled to a control hapten, TNBS (2,4,6-trinitrobenzenesulfonic acid, SIGMA), or of APCs coupled to DNBS, prepared from splenocytes of mice having received 100 μg of NP, 5×10⁶ MV-UV particles, 500 μl of dummy preparation or 500 μl of PBS buffer, by injection into the peritoneal cavity. The APCs were prepared according to the protocol described in example 4 above.

The cell cultures were prepared in RPMI 1640, 10% FCS, in 96-well plates. The CD8 T lymphocytes (CD8LTs) were cultured in a proportion of 5×10⁵ cells per well in the presence of 10⁶ APCs coupled or not coupled to a hapten. For each condition, the CD8LTs and the APCs were cultured separately, making it possible to ensure that there was no spontaneous proliferation of the CD8LTs and that the APCs had been correctly irradiated. The cell culture was carried out for 90 hours. After 72 hours, 0.5 μCi of tritiated thymidine were added to each well. The cells were transferred onto filters and the number of counts per minute was counted in the presence of scintillation fluid (WALLAC counter).

The hapten-specific proliferation indices were calculated according to the following formula:

[Cpm(CD8LTs+APCs coupled to the hapten)]/[Cpm−(CD8LTs+APCs not coupled to the hapten)]

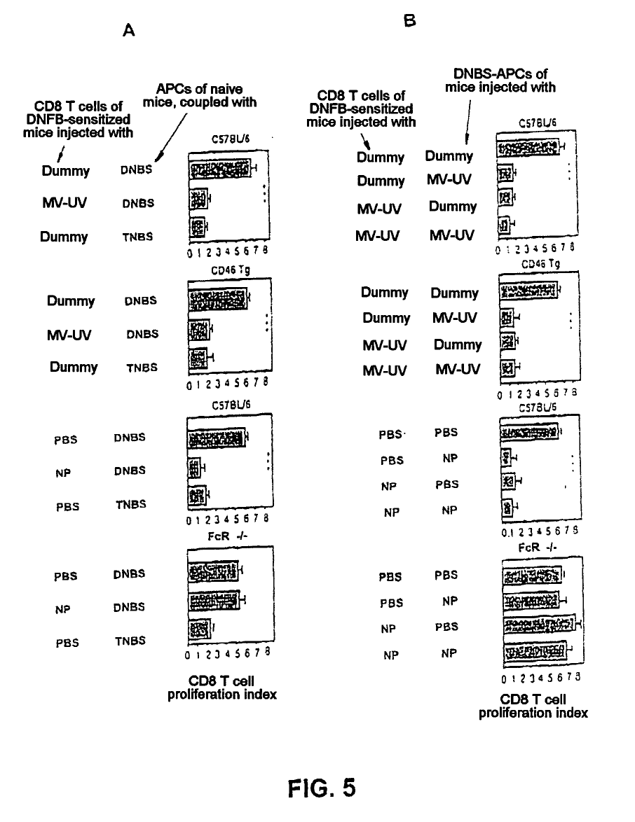

The values of the mean antigen-specific proliferation indices are represented in FIGS. 5(a) (APCs of naïve mice) and 5(b) (APCs of mice having received the same treatment as the CD8LT donors).

The results given in FIG. 5(a) show that the CD8LTs of mice injected with the dummy preparation and sensitized respond strongly to the APCs of naïve mice, coupled to DNBS, but not to the APCs of naïve mice, coupled to a control hapten, which shows the antigen-specific nature of the response. On the other hand, in the case of the mice injected with the preparation of MV-UV or of NP, an inhibition of the response of the CD8LTs of the C57BL/6 mice or of the CD46 mice to the APCs of naïve mice, coupled to DNBS, is observed. This inhibition is not observed in the case of the CD8LTs of the mice lacking the FcγR receptor.

The results given in FIG. 5(b) show that, in the presence of APCs originating from mice injected with MV-UV or NP, the antigen-specific proliferation of the CD8 T lymphocytes of C57BL/6 mice or mice transgenic to CD46, sensitized to DNVB, is inhibited.

This inhibition is not observed in the case of the CD8LTs of the mice lacking the FcγR receptor.

The CD8 T lymphocytes derived from sensitized mice having received an injection of MV-UV or NP exhibit a lack of CD8 T lymphocyte proliferation. In addition, the APCs derived from mice injected with MV-UV or NP are incapable of activating CD8 T lymphocytes in vitro.

These phenomena are independent of the expression of the CD46 molecule, but are not observed in mice deficient for the FcγR receptor, which indicates that MV-UV, and also NP, may impair the function of the APCs via this receptor, acting not only at the level of the phase of sensitization of naïve CD8 lymphocytes, but also at the level of the effector phase, following secondary contact with the antigen.

### EXAMPLE 6

### Effect of UV-Inactivated Measles Virus or of the Nucleoprotein NP on the Effector Phase of the Contact Hypersensitivity Reaction

This effect was tested on C57BL/6 mice, or on transgenic mice expressing the CD46 molecule.

The animals were sensitized to DNFB as described in example 1 above. 5 days later, 100 μg of NP, 5×10⁶ MV-UV particles, or 500 μl of dummy preparation were injected into the peritoneal cavity of the mice, and the application DNFB was applied 6 hours later, according to the protocol described in example 1 above.

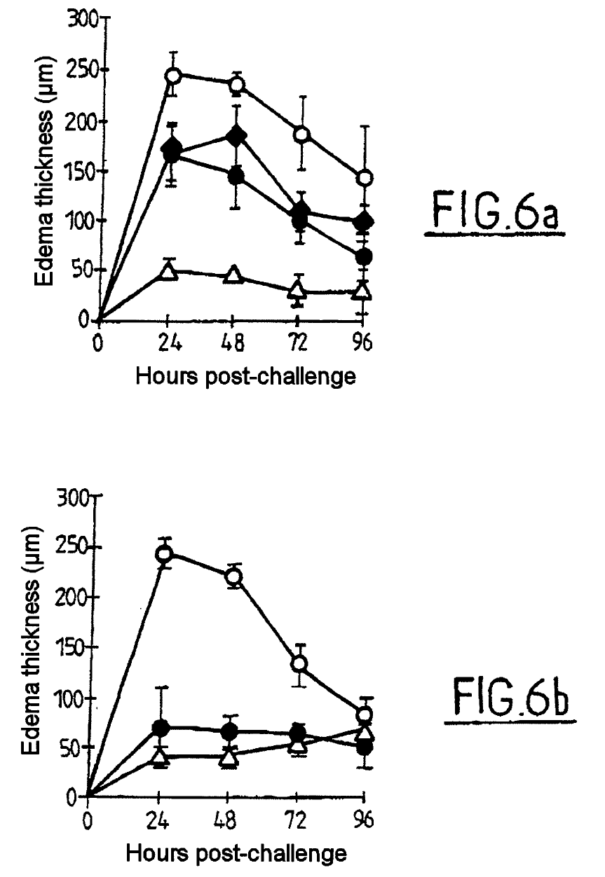

The edema thickness at various times following DNFB application was measured, and the results are given in FIGS. 6(a), for the C57BL/6 mice, and 6(b) for the mice transgenic for CD46.

Legend of FIGS. 6(a) and 6(b):

●: 5×10⁶ MV-UV

567 : recombinant NP

◯: dummy preparation

Δ: nonsensitized mice

In the C57BL/6 mice, a considerable inhibitory effect of MV-UV or NP is observed, similar to that observed in the case of an injection given before sensitization to DNFB (cf. example 2).

In the mice transgenic for CD46, the inhibitory effect of MV-UV is also similar to that observed in the case of an injection given before sensitization to DNFB (cf. example 3). Complete inhibition of the formation of the edema is observed.

These results show that the MV proteins are effective not only in the prevention, but also in the treatment, of an inflammatory response, and that, also in this case, expression of the CD46 receptor increases the immunosuppressive effect.

### EXAMPLE 7

### Assaying the MV Nucleoprotein NP by ELISA

The amount of nucleoprotein in viral preparations was measured by ELISA.

96-well plates were covered with anti-NP 33.4 antibody [LIBEAU et al., Vet. Rec., 134, 300-304, (1994)] in carbonate buffer (1/600 dilution of ascites fluids).

After overnight incubation at 4° C., the plates are blocked with PBS buffer, in 5% skimmed milk, and incubated again overnight at 4° C. with MV-UV supernatants. After addition of 1 μg/ml of biotinylated anti-NP antibody cl. 120 [GIRAUDON and WILD, J. Gen. Virol., 54, 325-332, (1981)], the plates are incubated with an ExtrAvidin® peroxydase conjugate diluted to 1/1000 (SIGMA). After addition of the substrate (ABTS; SIGMA), and incubation for 45 min, the OD is read at 400 nm. A standard curve is established with a series of dilutions of NP purified from Vero cells infected with MV.

### EXAMPLE 8

### Inhibition of Delayed Hypersensitivity Reactions by Proteins of Various Morbilliviruses

Inhibition of the DNFB Contact Hypersensitivity Reaction by UV-Inactivated Canine Distemper Virus

C57BL/6 mice, or mice lacking the FcγR receptor, were given intraperitoneal injections of 5×10⁶ particles of UV-inactivated canine distemper virus (CDV/UV), of 100 μg of MV NP, of 5×10⁶ MV-UV particles or 500 μl of PBS, and then sensitized to DNFB, according to the protocol described in example 1 above.

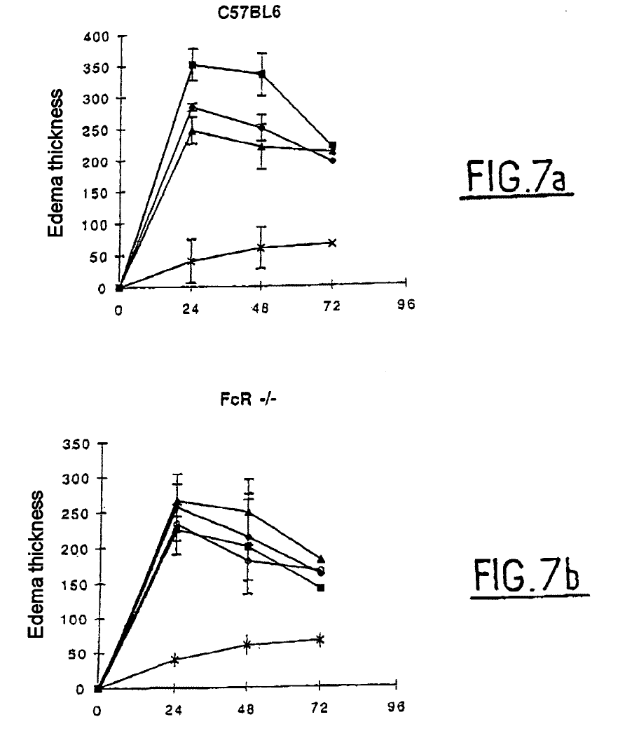

The edema thickness at various times following DNFB application was measured. The results are given in FIGS. 7(a) and 7(b):

FIG. 7(a): DNFB hypersensitivity test in C57BL/6 mice

◯: MV-UV

♦: CDV-UV

▴: recombinant NP

▪: PBS

\hat{9}: nonsensitized mice

FIG. 7(b): DNFB hypersensitivity test in FcγR^{-/-} mice.

◯: MV-UV

567 : CDV-UV

▴: recombinant NP

▪: PBS

\hat{9}: nonsensitized mice

These results show that the contact hypersensitivity reaction is significantly blocked by the UV-inactivated CDV virus (CDV-UV) (as much as with the nucleoprotein NP) in the normal C57BL/6 mice (FIG. 7a). On the other hand, neither the virus nor the NP are effective in the mice deficient for the FcγR receptor (FIG. 7b), indicating the need for FcγR for the immunosuppressive effect of morbilliviruses.

Inhibition of the KLH Delayed Hypersensitive Reaction by the NP

Recombinant preparations of peste des petits ruminants virus nucleoprotein (PPRVNP obtained from the sequence published by DIALLO et al.) or of the 125 C-terminal amino acids of measles virus nucleoprotein (MV-NPc) 35 were obtained as described in example 2. For the production of NPc, the construct encoding 125 C-terminal amino acids of measles virus nucleoprotein was cloned into the vector pQE-32 (QIAGEN), containing a histidine tag. The NPc+His Tag portion was then cloned into a baculovirus, which was used to infect Sf-9, as described in example 2. After culturing for three days, the cells were recovered. The NPc was purified from the cell pellet obtained with the QIAexpressionist® kit from QIAGEN, according to the protocol indicated by the manufacturer. C57BL/6 mice were given interperitoneal injections of 100 μg of PPRVNP, of 100 μg of MV-NPc, of 1 mg of dexamethasone or 500 μl of PBS, and were then sensitized to KLH according to the protocol described in example 1 above.

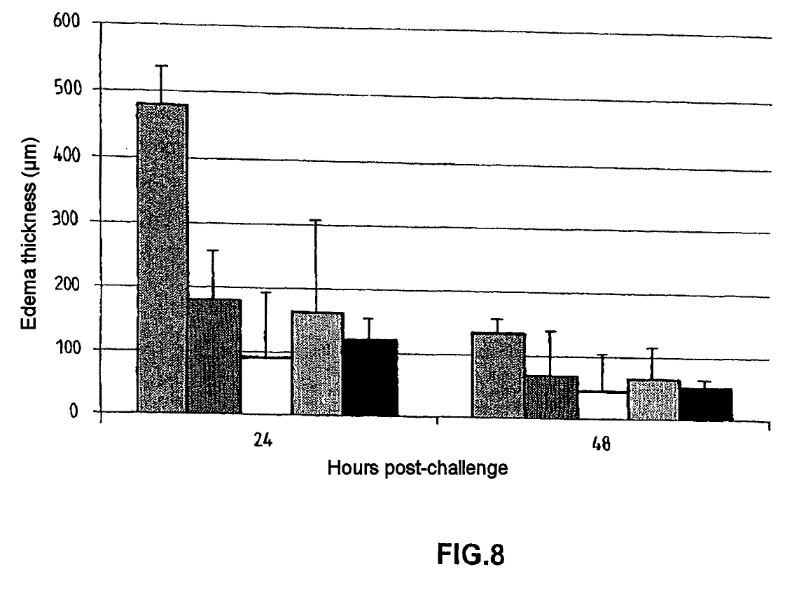

The results are given in FIG. 8, which represents the mean size of the pad edema at 24 hours and 48 hours after the boost injection.

Legend of FIG. 8:

Medium grey bars: PBS

Dark grey bars: PPRVNP

Light grey bars: dexamethasone

White bars: MV-NPv

Black bars: nonsensitized mice

These results show that the mice injected with PPRVNP, MV-NPc, or dexamethasone develop a delayed hypersensitivity response which is less substantial than the control mice injected with PBS.

The greatest inhibitory effect is observed with the MV-NPc. The inhibitory effect of the PPRVNP is similar to that of the dexamethasone.

### EXAMPLE 9

### Inhibition of the Mixed Lymphocyte Reaction by UV-Inactivated Measles Virus

The mixed lymphocyte reaction constitutes an in vitro model of allograft rejection.

BALB/c mice were injected intraperitoneally with 10⁵ MV-UV particles or 500 μl of dummy preparation. 48 hours later, splenocytes were prepared from the spleens of these mice, and stimulated, in mixed culture, with splenocytes obtained from C57BL/6 mice irradiated at 1500 rad.

Responder cells from BALB/c mice (5×10⁶/ml) were cultured with the same number of C57BL/6 stimulatory cells in RPMI medium supplemented with 10% FCS, 10 Mm HEPES, 2 Mm glutamine, 5×10⁶ M 2β-mercaptoethanol and 15 μg/ml of gentamycin, for 3 days at 37° C., 7% CO₂. The cell proliferation was evaluated on the 3^{rd} day, by incorporation of thymidine-3H (1 μCi/well) for 16 hours.

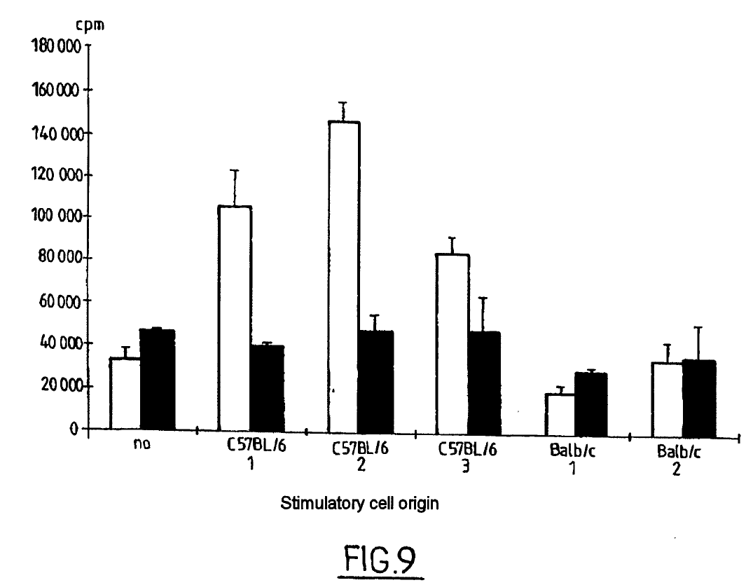

The results are given in FIG. 9. These results show inhibition, by the MV-UV, of the reaction of the lymphocytes from BALB/c mice in response to the stimulation by the cells of the C57BL/6 mice.

Legend of FIG. 9:

□dummy/preparation

▪: MV-UV

1. The use of a preparation of proteins comprising at least one morbillivirus nucleoprotein, or a fragment thereof comprising all or part of the approximately 100 to 130 amino acid C-terminal region of said nucleoprotein, for producing a medicinal product which inhibits the cell-mediated immune response.

2. The use as claimed in claim 1, characterized in that said fragment comprises at least one sequence of said C-terminal region which is conserved between the nucleoproteins of various morbilliviruses.

3. The use as claimed in claim 2, characterized in that said fragment is chosen from:
a fragment comprising amino acids 411-421 of the peptide sequence of the measles virus nucleoprotein, or the corresponding sequence of the nucleoprotein of another morbillivirus;
a fragment comprising amino acids 489-506 of the peptide sequence of the measles virus nucleoprotein, or the corresponding sequence of the nucleoprotein of another morbillivirus;
a fragment comprising amino acids 516-625 of the peptide sequence of the measles virus nucleoprotein, or the corresponding sequence of the nucleoprotein of another morbillivirus.

4. The use as claimed in any one of claims 1 to 3, characterized in that said preparation of proteins also comprises the envelope glycoproteins F and H of a morbillivirus.

5. The use as claimed in any one of claims 1 to 4, characterized in that said medicinal product is intended for the prevention or treatment of an inflammatory pathological condition.

6. The use as claimed in claim 5, characterized in that said medicinal product is intended for the prevention or treatment of allergic contact dermatitis.

7. Use as claimed in any one of claims 1 to 4, characterized in that said medicinal product is intended for the prevention or treatment of transplant rejection, or of graft versus host disease.

8. A method for evaluating the immunosuppressive potential of a preparation of a morbillivirus, characterized in that it comprises determining the amount of NP protein of said morbillivirus present in said preparation.

9. The method as claimed in claim 8, characterized in that it comprises bringing said preparation into contact with at least one antibody against said NP protein, and preferably at least one antibody against an epitope carried by the approximately 110 amino acid C-terminal region of said protein, and quantifying the antibody/antigen complex formed.

10. The use of mice sensitized to DNFB or to KLH, for evaluating the immunosuppressive potential of a preparation of proteins of a morbillivirus.

11. The use as claimed in claim 10, characterized in that said mice are transgenic mice expressing the CD46 receptor.

12. The use of a morbillivirus in which the 110 to 130 amino acid C-terminal region of the nucleoprotein has been deleted or altered, in at least one sequence of said C-terminal region which is conserved between the various morbilliviruses, for producing a vaccine.
